(19) United States
(12) Patent Application Publication
(10) Pub. No.: US 2025/0197357 A1
(43) Pub. Date: Jun. 19, 2025

(54) PPARG INVERSE AGONISTS AND USES THEREOF

(71) Applicant: Flare Therapeutics Inc., Cambridge, MA (US)

(72) Inventor: Jonathan E. Wilson, Arlington, MA (US)

(21) Appl. No.: 18/844,421

(22) PCT Filed: Mar. 3, 2023
(86) PCT No.: PCT/US2023/063650

§ 371 Date: Sep. 6, 2024

Related U.S. Application Data
(60) Provisional application No. 63/317,737, filed on Mar. 8, 2022.

(51) Int. Cl. C07D 231/12; A61K 31/166; A61K 31/44; A61P 35/00; C07C 317/32; C07D 213/84; C07D 401/12

(52) U.S. Cl. CPC C07D 231/12; A61K 31/166; A61K 31/44; A61P 35/00; C07C 317/32; C07D 213/84; C07D 401/12

(57) ABSTRACT

Provided are compounds of Formula (I); and pharmaceutically acceptable salts and compositions thereof, which are useful for treating a variety of conditions associated with PPARG.

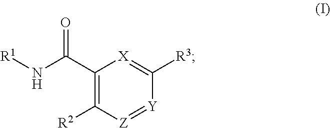

### RELATED APPLICATION

This application claims the benefit of priority to U.S. Provisional Application No. 63/317,737 filed Mar. 8, 2022, the entire contents of which are incorporated herein by reference.

### BACKGROUND

PPARgamma (PPARG) is a type II ligand-dependent nuclear hormone receptor (belonging to the PPAR nuclear receptor subfamily) that functions as an obligate heterodimer with retinoid X receptors (RXRs). PPARG is predominantly expressed in adipose tissue, colon, macrophages and the luminal layers of the urothelium. PPARG is known as a master regulator of adipogenesis, functioning to regulate adipocyte differentiation, fatty acid storage and glucose metabolism. PPARG has also been shown to play an important role in the metabolism and inflammation of macrophages, where it is induced by IL4 and controls glutamine metabolism. In the normal urothelium, PPARG is critical for its homeostasis and regeneration.

The role for PPARG in cancer was originally inferred from genomic studies that identified a PAX8-PPARG chromosomal rearrangement in follicular thyroid carcinomas. More recently, PPARG has been found to be over-expressed and genetically altered in the luminal subtype of urothelial cancer. This is consistent with reports that long-term use of PPARG agonists is associated with an increased incidence of urothelial cancer. Most urothelial cancers are urothelial carcinoma, which are classified as either non-muscle-invasive urothelial cancer (NMIUC, 70%), muscle-invasive urothelial cancer (MIUC, 25%) or metastatic urothelial cancer (MUC, 5%). MIUC is usually diagnosed de novo but may arise from the 10 to 20% of NMIUC cases that eventually progress. MIUC is a heterogeneous and aggressive disease, associated with a five-year survival rate of 60% for patients with localized disease and less than 10% for patients with distant metastases. Molecular understanding of NMIUC and MIUC has improved significantly, including the association between molecular subtypes and urothelial differentiation. Several molecular classes of MIUC have been proposed, whereby an activated PPARG signature features prominently in the luminal subtypes. First-line treatment is chemotherapy with several options in chemo-ineligible or second line, but treatment options are limited with poor overall survival rates.

The need exists to develop effective PPARG modulators for treating cancers such as NMIUC, MIUC, and MUC, and related conditions.

### SUMMARY

Provided herein are compounds having the Formula I:

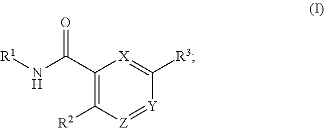

and pharmaceutically acceptable salts and compositions thereof, wherein R¹, R², R³, X, Y, and Z are as described herein. In one aspect, the disclosed compounds of Formula I and pharmaceutically acceptable salts thereof modulate PPARG (e.g., as agonists such as inverse agonists, and are useful in a variety of therapeutic applications such as, for example, in treating cancer. As such, their uses for treating diseases responsive to the inhibition of PPARG are included.

Pharmaceutical compositions comprising the compounds and pharmaceutically acceptable salts of the disclosed compounds of Formula I, as well as methods for their preparation are also included.

### DETAILED DESCRIPTION

### 1. General Description of Compounds

In a first embodiment, provided herein is a compound of Formula I:

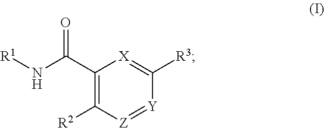

X, Y, and Z are each independently selected from N and —CR⁴;
   R¹ is selected from phenyl, heterocyclyl, and heteroaryl, each of which are optionally substituted with 1 to 3 groups selected from R⁵;
   R² is selected from halo, —SR^{a}, —SOR^{a}, —SO₂R^{a}, —OR^{a}, and —SO(═NR^{a})R^{b};
   R³ is selected from cyano and nitro;
   R⁴ is selected from hydrogen, halo, (C₁-C₄)alkyl, halo(C₁-C₄)alkyl, (C₁-C₄)alkoxy, and hydroxyl;
   R⁵ is selected from halo, (C₁-C₄)alkyl, (C₁-C₄)alkoxy, halo(C₁-C₄)alkyl, halo(C₁-C₄)alkoxy, cyano, oxo, —(C₁-C₄)alkylOR^{a}, —(C₁-C₄)alkylC(O)R^{a}, —(C₁-C₄)alkylC(O)OR^{a}, —C(O)NR^{a}R^{b}, —(C₁-C₄)alkylC(O)NR^{a}R^{b}, —C(O)R^{a}, —C(O)OR^{a}, —NR^{a}R^{b}, —(C₁-C₄)alkylNR^{a}R^{b}, —C(O)NR^{a} SO₃H, —NR^{a} C(O)R^{b}, —NR^{a} C(O)OR^{b}, —NR^{a}C(S)OR^{b}, —NR^{c}C(O)N^{a}R^{b}, —NR^{c}C(S)NR^{a}R^{b}, —NR^{c}S(O)₂NR^{a}R^{b}, —C(S)R^{a}, —S(O)₂R^{a}, —S(O)R^{a}, —C(S)OR^{a}, —C(S)NR^{a}R^{b}, —NR^{a} C(S)R^{b}, —SR^{a}, —O (phenyl), phenyl, heterocyclyl, and heteroaryl, wherein each of said phenyl, heterocyclyl, heteroaryl, and the phenyl group on-O (phenyl) are optionally and independently substituted with 1 to 3 groups selected from R⁶;
   R⁶ is selected from halo, (C₁-C₄)alkyl, halo(C₁-C₄)alkyl, (C₁-C₄)alkoxy, halo(C₁-C₄)alkoxy, nitro, oxo, cyano, —(C₁-C₄)alkylOR^{d}, —(C₁-C₄)alkylC(O)R^{d}, —(C₁-C₄)alkylC(O)OR^{d}, —C(O)NR^{d}R^{e}, —(C₁-C₄)alkylC(O)NR^{d}R^{e}, —C(O)R^{d}, —C(O)OR^{d}, —NR^{d}R^{e}, —(C₁-C₄)alkylNR^{d}R^{e}, —C(O)NR^{a} SO₃H, —NR^{d}C(O)R^{e}, —NR^{d}C(O)OR^{e}, —NR^{d}C(S)OR^{e}, —NR^{f}C(O)N^{d}R^{e}, —NR^{f}C(S)NR^{d}R^{e}, —NR^{f}S(O)₂NR^{d}R^{e}, —C(S)R^{d}, —S(O)₂R^{d}, —S(O)R^{d}, —C(S)OR^{d}, —C(S)NR^{d}R^{e}, —NR^{d}C(S)R^{e}, and —SR^{d}; and
   R^{a}, R^{b}, R^{c}, R^{d}, and R^{e} are each independently selected from hydrogen, (C₁-C₄)alkyl, and halo(C₁-C₄)alkyl.

### 2. Definitions

When used in connection to describe a chemical group that may have multiple points of attachment, a hyphen (-) designates the point of attachment of that group to the variable to which it is defined. For example, —NR^{b}C(O)OR^{c} and —NR^{b}C(S)OR^{c} mean that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, and the like, means saturated straight-chain or branched monovalent hydrocarbon radical.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., —OCHF₂ or —OCF₃.

The term oxo means the group ═O.

The term “heteroaryl” used alone or as part of a larger moiety refers to a 5- to 12-membered aromatic radical containing 1-4 heteroatoms selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 5- to 12-membered saturated or partially unsaturated heterocyclic ring containing 1 to 4 heteroatoms independently selected from N, O, and S. It can be monocyclic, bicyclic (e.g., a bridged, fused, or spiro bicyclic ring), or tricyclic. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. A heterocyclyl group may be mono- or bicyclic. The term “heterocyclyl” also includes, e.g., unsaturated heterocyclic radicals fused to another unsaturated heterocyclic radical or aryl or heteroaryl ring, such as for example, tetrahydronaphthyridine, indolinone, dihydropyrrolotriazole, imidazopyrimidine, quinolinone, dioxaspirodecane. It will also be understood that when specified, optional substituents on a heterocyclyl group may be present on any substitutable position and, include, e.g., the position at which the heterocyclyl is attached (e.g., in the case of an optionally substituted heterocyclyl or heterocyclyl which is optionally substituted).

The term “spiro” refers to two rings that shares one ring atom (e.g., carbon).

The term “fused” refers to two rings that share two adjacent ring atoms with one another.

The term “bridged” refers to two rings that share three ring atoms with one another.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that will elicit a desired or beneficial biological or medical response of a subject e.g., a dosage of between 0.01-100 mg/kg body weight/day.

### 3. Compounds

In a second embodiment, X, Y, and Z in the compound of Formula I, or a pharmaceutically acceptable salt thereof, are each —CR⁴; or X and Z are each —CR⁵ and Y is N, wherein the remaining variables are as described above for Formula I. Alternatively, as part of a second embodiment, X, Y, and Z in the compound of Formula I, or a pharmaceutically acceptable salt thereof, are each —CR⁴, wherein the remaining variables are as described above for Formula I.

In a third embodiment, R⁴ in the compound of Formula I, or a pharmaceutically acceptable salt thereof, is selected from hydrogen, (C₁-C₄)alkyl, halo(C₁-C₄)alkyl, and (C₁-C₄)alkoxy, wherein the remaining variables are as described above for Formula I or the second embodiment. Alternatively, as part of a third embodiment, R⁴ in the compound of Formula I, or a pharmaceutically acceptable salt thereof, is hydrogen, wherein the remaining variables are as described above for Formula I or the second embodiment.

In a fourth embodiment, R³ in the compound of Formula I, or a pharmaceutically acceptable salt thereof, is cyano, wherein the remaining variables are as described above for Formula I or the second or third embodiment.

In a fifth embodiment, R² in the compound of Formula I, or a pharmaceutically acceptable salt thereof, is selected from halo, —SR^{a}, —SOR^{a}, —SO₂R^{a}, and —SO(═NR^{a})R^{b}, wherein the remaining variables are as described above for Formula I or the second, third, or fourth embodiment. Alternatively, as part of a fifth embodiment, R² in the compound of Formula I, or a pharmaceutically acceptable salt thereof, is selected from —SO₂R^{a}, and —SR^{a}, wherein the remaining variables are as described above for Formula I or the second, third, or fourth embodiment. In another alternative, as part of a fifth embodiment, R² in the compound of Formula I, or a pharmaceutically acceptable salt thereof, is selected from —SO₂Me, SCF₃, —SO₂CH₂CF₃, and —SO₂CF₃, wherein the remaining variables are as described above for Formula I or the second, third, or fourth embodiment.

In a sixth embodiment, R¹ in the compound of Formula I, or a pharmaceutically acceptable salt thereof, is selected from phenyl and heteroaryl, each of which are optionally substituted with 1 to 3 groups selected from R⁵, wherein the remaining variables are as described above for Formula I or the second, third, fourth, or fifth embodiment. Alternatively, as part of a sixth embodiment, R¹ in the compound of Formula I, or a pharmaceutically acceptable salt thereof, is selected from phenyl, pyridinyl, isoquinolinyl, pyrazolopyridinyl, each of which are optionally substituted with 1 to 3 groups selected from R⁵, wherein the remaining variables are as described above for Formula I or the second, third, fourth, or fifth embodiment.

In a seventh embodiment, R⁵ in the compound of Formula I, or a pharmaceutically acceptable salt thereof, is selected from halo, oxo, cyano, (C₁-C₄)alkyl, (C₁-C₄)alkoxy, halo(C₁-C₄)alkyl, halo(C₁-C₄)alkoxy, —(C₁-C₄)alkylOR^{a}, —(C₁-C₄)alkylC(O)R^{a}, —(C₁-C₄)alkylC(O)OR^{a}, —C(O)NR^{a}R^{b}, —(C₁-C₄)alkylC(O)NR^{a}R^{b}, —C(O)R^{a}, —C(O)OR^{a}, —NR^{a}R^{b}, —(C₁-C₄)alkylNR^{a}R^{b}, NR^{a}C(O)R^{b}, —NR^{a}C(O)OR^{b}, —NR^{c}C(O)N^{a}R^{b}, —NR^{c}S(O)₂NR^{a}R^{b}, —S(O)₂R^{a}, —S(O)R^{a}, —SR^{a}, —O(phenyl), phenyl, heterocyclyl, and heteroaryl, wherein each of said phenyl, heterocyclyl, heteroaryl, and the phenyl group on —O(phenyl) are optionally and independently substituted with 1 to 3 groups selected from R⁶, wherein the remaining variables are as described above for Formula I or the second, third, fourth, fifth, or sixth embodiment. Alternatively, as part of a seventh embodiment, R⁵ in the compound of Formula I, or a pharmaceutically acceptable salt thereof, is selected from halo, halo(C₁-C₄)alkyl, cyano, oxo, —O(phenyl), heterocyclyl, and heteroaryl, wherein each of said heterocyclyl, heteroaryl, and the phenyl group on —O(phenyl) are optionally and independently substituted with 1 to 3 groups selected from R⁶, wherein the remaining variables are as described above for Formula I or the second, third, fourth, fifth, or sixth embodiment. In another alternative, as part of a seventh embodiment, R⁵ in the compound of Formula I, or a pharmaceutically acceptable salt thereof, is selected from cyano, chloro, fluoro, CF₃, oxo, methyl, pyridinyl, piperazinyl, pyrazolyl, and —O(phenyl), wherein the remaining variables are as described above for Formula I or the second, third, fourth, fifth, or sixth embodiment.

In an eighth embodiment, R⁶ in the compound of Formula I, or a pharmaceutically acceptable salt thereof, is selected from halo, (C₁-C₄)alkyl, halo(C₁-C₄)alkyl, (C₁-C₄)alkoxy, halo(C₁-C₄)alkoxy, oxo, cyano, —(C₁-C₄)alkylOR^{d}, —(C₁-C₄)alkylC(O)OR^{d}, —NR^{d}R^{e}, —(C₁-C₄)alkylNR^{d}R^{c}, —C(O)NR^{d}R^{c}, —(C₁-C₄)alkylC(O)NR^{d}R^{e}, —C(O)R^{d}, —C(O)OR^{d}, —S(O)₂R^{d}, —S(O)R^{d}, and —SR^{d}, wherein the remaining variables are as described above for Formula I or the second, third, fourth, fifth, sixth, or seventh embodiment. Alternatively, as part of an eighth embodiment, R⁶ in the compound of Formula I, or a pharmaceutically acceptable salt thereof, is selected from halo, (C₁-C₄)alkyl, halo(C₁-C₄)alkyl, oxo, —C(O)R^{d}, wherein the remaining variables are as described above for Formula I or the second, third, fourth, fifth, sixth, or seventh embodiment. In another alternative, as part of an eighth embodiment, R⁶ in the compound of Formula I, or a pharmaceutically acceptable salt thereof, is selected from methyl, CF₃, —CHCF₂, oxo, chloro, and C(O)CH₃, wherein the remaining variables are as described above for Formula I or the second, third, fourth, fifth, sixth, or seventh embodiment.

Compounds having the Formula I are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of PPARG. In some aspects, the compounds, pharmaceutical acceptable salts, and pharmaceutical compositions described herein inhibit the activity PPARG. In some aspects, the compounds and pharmaceutical acceptable salts disclosed herein are agonists of PPARG. In some aspects, the compounds and pharmaceutical acceptable salts disclosed herein are agonists of PPARG. In some aspects, the compounds and pharmaceutical acceptable salts disclosed herein are inverse agonists of PPARG. In one aspect, “inverse-agonists” refer to agents that bind to the same receptor binding site as a agonist (e.g., the binding site of a nuclear receptor such as PPARG) and not only antagonizes the effects of an agonist but, moreover, exerts the opposite effect by suppressing spontaneous receptor signaling (when present).

In some aspects, the compounds and pharmaceutical acceptable salts disclosed herein overcome the activated state of PPARG function resulting from alteration in PPARG activity (mutation, amplification or overexpression) or from RXRA activating mutations. In some aspect, the compounds and pharmaceutical acceptable salts disclosed herein increase the repressive state (NCOR1 recruitment) to a higher degree than previously disclosed PPARG modulators such as prior inverse agonists. Such results even arise in the mutant context. See e.g., the table qualitatively assessing NCOR1 recruitment and repression of PPARG target genes in HT1197 in the Exemplification section.

In some aspects, the compounds and pharmaceutical compositions described herein are useful in treating a disorder associated with PPARG function. Thus, provided herein are methods of treating a disorder associated with PPARG function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a disorder associated with PPARG function. Also provided is a compound described herein, or a pharmaceutically acceptable salt thereof, or a pharmaceutical composition comprising a disclosed compound or pharmaceutically acceptable salt thereof, for use in treating a disorder associated with PPARG.

In one aspect, the disorder associated with PPARG is cancer. In some aspects, the cancer is associated with an up-regulated peroxisome proliferator-activated receptor (PPAR) signaling pathway. In some aspects, the up-regulated PPAR signaling pathway is associated with increased expression of one or more genes selected from Uroplakin 1A (UPK1A), Uroplakin IB (UPK1B), Uroplakin (UPK2), Keratin 20 (KRT20), GATA Binding Protein 3 (GAT A3), Nuclear Receptor Corepressor 1 (NCOR1), Nuclear Receptor Corepressor 2 (NCOR2), Fatty Acid Binding Protein 4 (FABP4), Forkhead Box A1 (FOXA1), CD36 Molecule (CD36), Acyl-CoA Oxidase 1 (ACOX1), 3-Hydroxy-3-Methylglutaryl-CoA Synthase 2 (HMGCS2), Acyl-CoA Synthetase Long-Chain Family Member 5 (ACSL5), Arachidonate 5-Lipoxygenase (ALOX5), Acyl-CoA Synthetase Long-Chain Family Member 1 (ACSL1), and Angiopoietin Like 4 (ANGPTL4).

In some aspects, the cancer treated by the compounds, pharmaceutically acceptable salt thereof, and pharmaceutical compositions described herein is selected from breast cancer, pancreatic cancer, ovarian cancer, prostate cancer, renal cancer, bladder cancer, testicular cancer, urothelial cancer (e.g., non-muscle-invasive urothelial cancer, muscle-invasive urothelial cancer, metastatic urothelial cancer), skin cancer, melanoma, colon cancer, kidney cancer, brain cancer and a hematopoietic cancer (e.g., lymphoma, multiple myeloma and leukemia). In one aspect, the cancer treated by the compounds, pharmaceutically acceptable salt thereof, and pharmaceutical compositions described herein is urothelial cancer such as non-muscle-invasive urothelial cancer, muscle-invasive urothelial cancer, and metastatic urothelial cancer.

Other uses besides cancer are contemplated and include e.g., metabolic diseases (e.g., osteoporosis, rachitis, arthrosis, obesity, type I and type II diabetes mellitus), lipid metabolism disorder, pancreatitis, glucose metabolism disorder, diabetic neuropathy, diabetic complications, hyperuricemia, osteoporosis, rachitis, arthrosis inflammatory diseases (e.g., inflammatory skin diseases such as psoriasis, atopic dermatitis, eczema, acne vulgaris, other dermatitides and pruritus), pulmonary disorders (e.g., asthma and chronic obstructive pulmonary disease), autoimmune disease, neurodegenerative disease (e.g., multiple sclerosis, Alzheimer's disease, and Parkinson's disease), cardiovascular diseases (e.g., selected from atherosclerosis, venous and arterial occlusive diseases), restenosis after invasive procedures, cardiomyopathy, myocardial fibrosis, congestive heart failure, angiogenesis and neovascularization in neoplastic diseases and renal diseases.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### Exemplification

### Chemical Synthesis

The representative examples that follow are intended to help illustrate the present disclosure, and are not intended to, nor should they be construed to, limit the scope of the invention.

### Starting Materials

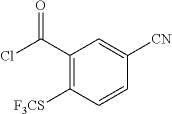

### 5-cyano-2-(trifluoromethylsulfanyl)benzoyl chloride

Step 1, methyl 5-cyano-2-(trifluoromethylsulfanyl)benzoate: To a solution of methyl 5-cyano-2-iodo-benzoate (3.0 g, 10.5 mmol, 1.0 equiv.) and trifluoromethylsulfanylsilver (2.18 g, 10.5 mmol, 1.0 equiv.) in DMF (30 mL) was added CuBr (150 mg, 1.05 mmol, 0.1 equiv.) and 1,10-phenanthroline (377 mg, 2.09 mmol, 0.2 equiv.). The mixture was stirred at 80° C. for 12 hours. The reaction mixture was diluted with water (60 mL) and the mixture was extracted with ethyl acetate (3×30 mL). The combined organic layers were washed with brine (2×25 mL), dried over Na₂SO₄, filtered, and concentrated under reduced pressure. The residue was purified by silica gel column chromatography (13:1 petroleum ether:ethyl acetate) to afford the title compound (1.22 g, 43% yield) as a white solid. ¹H NMR (400 MHz, CHLOROFORM-d) δ=8.31 (d, J=1.6 Hz, 1H), 7.86-7.82 (m, 1H), 7.81-7.78 (m, 1H), 4.00 (s, 3H).

Step 2, 5-cyano-2-(trifluoromethylsulfanyl)benzoic acid: To a solution of methyl 5-cyano-2-(trifluoromethylsulfanyl)benzoate (1.2 g, 4.59 mmol, 1.0 equiv.) in THF (12 mL) was added a solution of LiOH·H₂O (193 mg, 4.59 mmol, 1.0 equiv.) in water (3 mL). The reaction mixture was stirred at 20° C. for 2 hours. The pH of the reaction mixture was adjusted to 2-3 with HCl (2M). The mixture was filtered and the filtered cake was washed with water (2×100 mL) and dried in vacuum to afford the title compound (1.02 g, 4.13 mmol, 90.0% yield) as a yellow solid. LCMS [M−1]=246.0. ¹H NMR (400 MHz, DMSO-d6) δ=8.40 (d, J=2.0 Hz, 1H), 8.14 (dd, J=2.0, 8.4 Hz, 1H), 7.85 (s, 1H).

Step 3. 5-cyano-2-(trifluoromethylsulfanyl)benzoyl chloride: A mixture of 5-cyano-2-(trifluoromethylsulfanyl)benzoic acid (210 mg, 849.54 μmol, 1 equiv.) in SOCl₂ (2 mL) was degassed with N₂ and then the mixture was stirred at 80° C. for 2 hours under N₂. The reaction mixture was concentrated under reduced pressure to afford the title compound (226 mg, crude) as light white solid.

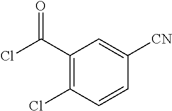 [SMILES: N#Cc1ccc(Cl)c(C(=O)Cl)c1]

2-chloro-5-cyano-benzoyl chloride: SOCl₂ (1.0 mL) was added to a solution of 2-chloro-5-cyanobenzoic acid (75.0 mg, 413 μmol, 1.0 equiv.). The mixture solution was stirred at 80° C. for 0.5 hours. The solution was concentrated under reduced pressure to afford the title compound (83.0 mg, crude) as a white solid. The product was used directly in next step.

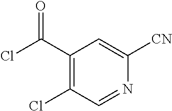 [SMILES: N#Cc1cc(C(=O)Cl)c(Cl)cn1]

5-chloro-2-cyano-pyridine-4-carbonyl chloride: SOCl₂ (15 mL) was added to a solution of 5-chloro-2-cyano-pyridine-4-carboxylic acid (1.45 g, 7.94 mmol, 1 equiv.) and the mixture was stirred at 100° C. for 2 hours. The mixture was concentrated to afford the title compound (1.59 g, 99% yield) as a white solid, which was used in the next step directly.

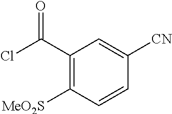 [SMILES: CS(=O)(=O)c1ccc(C#N)cc1C(=O)Cl]

### 5-cyano-2-methylsulfonyl-benzoyl chloride

Step 1, 5-cyano-2-(methylsulfonyl)benzoic acid: To a solution of 2-chloro-5-cyanobenzoic acid (1.0 g, 5.51 mmol, 1.0 equiv.) in DMF (4 mL) was added CuI (105 mg, 551 μmol, 0.1 equiv.), K₃PO₄ (1.75 g, 8.27 mmol, 1.5 equiv.) and sodium methanesulfinate (843 mg, 8.27 mmol, 1.5 equiv.). The mixture was stirred at 100° C. for 16 hours. The reaction mixture was quenched by the addition of HCl (1M, 24 mL) at 15° C. The mixture was filtered and concentrated under reduced pressure to afford the title compound (713 mg, 3.17 mmol, 57.4% yield) as a brown solid. ¹H NMR (400 MHz, DMSO-d₆) δ 14.52-13.87 (m, 1H), 8.57-7.88 (m, 3H), 3.46 (br s, 3H).

Step 2, 5-cyano-2-methylsulfonyl-benzoyl chloride: A solution of 5-cyano-2-(methylsulfonyl)benzoic acid (100 mg, 444 umol, 1.0 equiv.) in SOCl₂ (1.5 mL) was stirred at 80° C. for 1 hour. The reaction mixture was concentrated under reduced pressure to afford the title compound (108 mg, crude) as a brown solid. This material was used in the next step without further purification.

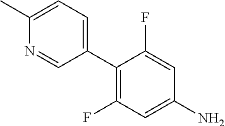 [SMILES: Cc1ccc(-c2c(F)cc(N)cc2F)cn1]

### 3,5-difluoro-4-(6-methylpyridin-3-yl)aniline

To a solution of 2-methyl-5-(4,4,5,5-tetramethyl-1,3,2-dioxaborolan-2-yl)pyridine (0.2 g, 913 μmol, 1.0 equiv.) in dioxane (6.0 mL) and water (2.0 mL) was added 4-bromo-3,5-difluoroaniline (233 mg, 913 μmol, 1.0 equiv.), K₂CO₃ (379 mg, 2.7 mmol, 3.0 equiv.) and Pd(dppf)Cl₂·CH₂Cl₂ (74.5 mg, 91.2 μmol, 0.1 equiv.). The mixture was degassed with N₂, and then stirred at 80° C. for 16 hours under N₂. The reaction mixture was cooled to rt, diluted with water (20 mL) and extracted with ethyl acetate (3×10 mL). The combined organic layers were washed with brine (20 mL), dried over Na₂SO₄, filtered, and concentrated under reduced pressure. The residue was purified by silica gel column chromatography (5:1 to 4:1 petroleum ether:ethyl acetate) to afford the title compound (144 mg, 70.8% yield, 98.9% purity) as a yellow solid. ¹H NMR (400 MHz, CHLOROFORM-d) δ 8.56 (s, 1H), 7.64 (br d, J=8.0 Hz, 1H), 7.21 (d, J=8.0 Hz, 1H), 6.41-6.19 (m, 2H), 3.98 (br s, 2H), 2.60 (s, 3H).

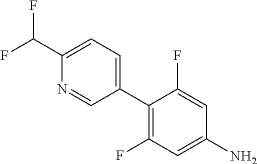 [SMILES: Nc1cc(F)c(-c2ccc(C(F)F)nc2)c(F)c1]

### 4-(6-(difluoromethyl)pyridin-3-yl)-3,5-difluoroaniline

The title compound was prepared in a similar manner to that described for 3,5-difluoro-4-(6-methylpyridin-3-yl)aniline using 2-difluoromethyl-5-(4,4,5,5-tetramethyl-1,3,2-dioxaborolan-2-yl)pyridine as a starting material. ¹H NMR (400 MHz, CHLOROFORM-d) δ 8.72 (s, 1H), 7.91 (br d, J=7.6 Hz, 1H), 7.69 (d, J=8.0 Hz, 1H), 6.87-6.51 (t, J=55.2 Hz, 1H), 6.38-6.23 (m, 2H), 4.27-3.82 (m, 2H).

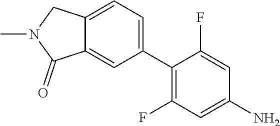 [SMILES: CN1Cc2ccc(-c3c(F)cc(N)cc3F)cc2C1=O]

### 6-(4-amino-2,6-difluorophenyl)isoindolin-1-one

The title compound was prepared in a similar manner to that described for 3,5-difluoro-4-(6-methylpyridin-3-yl)aniline using 2-methyl-6-(4,4,5,5-tetramethyl-1,3,2-dioxaborolan-2-yl)isoindolin-1-one as a starting material. ¹H NMR (400 MHz, METHANOL-d₄) δ 7.74 (s, 1H), 7.62-7.54 (m, 2H), 6.37-6.27 (m, 2H), 4.55-4.50 (m, 2H), 3.21 (s, 3H).

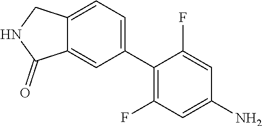 [SMILES: Nc1cc(F)c(-c2ccc3c(c2)C(=O)NC3)c(F)c1]

### 6-(4-amino-2,6-difluorophenyl)-2-methylisoindolin-1-one

The title compound was prepared in a similar manner to that described for 3,5-difluoro-4-(6-methylpyridin-3-yl)aniline using 6-(4,4,5,5-tetramethyl-1,3,2-dioxaborolan-2-yl)isoindolin-1-one as a starting material. ¹H NMR (400 MHz, DMSO-d₆) δ 8.60 (s, 1H), 7.79-7.43 (m, 3H), 6.43-6.15 (m, 2H), 5.89 (s, 2H), 4.40 (s, 2H).

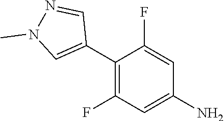

### 3,5-difluoro-4-(1-methyl-1H-pyrazol-4-yl)aniline

The title compound was prepared in a similar manner to that described for 3,5-difluoro-4-(6-methylpyridin-3-yl)aniline using 1-methyl-4-(4,4,5,5-tetramethyl-1,3,2-dioxaborolan-2-yl)-1H-pyrazole as a starting material. LCMS: [M+1]=210.1. ¹H NMR (400 MHz, DMSO-d₆) δ 7.86 (s, 1H), 7.60 (s, 1H), 6.27 (br d, J=11.4 Hz, 2H), 5.70 (s, 2H), 3.86 (s, 3H).

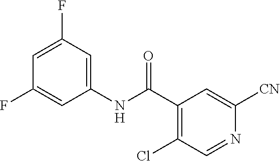 [SMILES: N#Cc1cc(C(=O)Nc2cc(F)cc(F)c2)c(Cl)cn1]

### 5-chloro-2-cyano-N-(3,5-difluorophenyl)isonicotinamide

A solution of 5-chloro-2-cyano-pyridine-4-carbonyl chloride (311 mg, 1.55 mmol, 1.0 equiv.) in isopropyl acetate (2 mL) was added to a solution of 3,5-difluoroaniline (200 mg, 1.55 mmol, 1.0 eq) in isopropyl acetate (2 mL) at 20° C. The mixture was stirred at 80° C. for 1 hour. The reaction mixture was partitioned between ethyl acetate (30 mL) and water (40 mL). The organic phase was separated, washed with brine (20 mL), dried over Na₂SO₄, filtered, and concentrated under reduced pressure. The crude product was triturated with ethyl acetate (3 mL) at 25° C. to afford the title compound (178 mg, 39% yield) as a white solid. LCMS [M−1]=292.0. ¹H NMR (METHANOL-d4, 400 MHz) δ 8.8-8.9 (m, 1H), 8.15 (s, 1H), 7.3-7.4 (m, 2H), 6.7-6.8 (m, 1H).

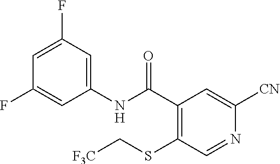 [SMILES: N#Cc1cc(C(=O)Nc2cc(F)cc(F)c2)c(SCC(F)(F)F)cn1]

### 2-cyano-N-(3,5-difluorophenyl)-5-((2,2,2-trifluoroethyl)thio)isonicotinamide

To a solution of 2,2,2-trifluoroethane-1-thiol (75.5 uL, 851 umol, 2.5 equiv.) in DMF (1 mL) was added NaH (34.1 mg, 851 umol, 2.5 equiv.; 60% dispersion in oil) at 0° C. The mixture was stirred at 0° C. for 20 minutes, and then 5-chloro-2-cyano-N-(3,5-difluorophenyl)isonicotinamide (100 mg, 340 umol, 1.0 equiv.) was added to the mixture at 0° C. The resulting mixture was stirred at 0° C. for 1 hour. The mixture was added to water (5 mL). The resulting suspension was filtered and the filter cake was washed with water (3×10 mL). The solid was concentrated under reduced pressure to afford the title compound (120 mg, 94% yield) as white solid. LCMS [M+1]=374.1.

¹H NMR (400 MHz, DMSO-d₆) δ=11.12 (s, 1H), 9.10 (s, 1H), 8.35 (s, 1H), 7.41 (dd, J=2.0, 9.0 Hz, 2H), 7.13-7.03 (m, 1H), 4.39 (q, J=10.2 Hz, 2H).

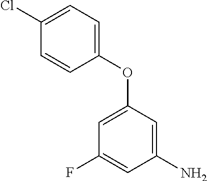 [SMILES: Nc1cc(F)cc(Oc2ccc(Cl)cc2)c1]

### 3-(4-chlorophenoxy)-5-fluoroaniline

Step 1, 1-(4-chlorophenoxy)-3-fluoro-5-nitrobenzene: A mixture of 4-chlorophenol (2.4 g, 18.9 μmol, 1.0 equiv.), 1,3-difluoro-5-nitrobenzene (3.0 g, 18.9 μmol, 1.0 equiv.) and Cs₂CO₃ (9.2 g, 28.3 μmol, 1.5 equiv.) in DMA (30 mL) was degassed with N₂. Then the mixture was stirred at 65° C. for 8 hours under N₂. The reaction mixture was diluted with water (200 mL) and extracted with 3thyl acetate (3×200 mL). The combined organic layers were washed with brine (3×100 mL), dried over Na₂SO₄, filtered, and concentrated under reduced pressure. The residue was purified by silica gel column chromatography (0-50% ethyl acetate in petroleum ether:ethyl acetate) to afford the title compound (2.96 g, 59% yield) as a white solid. ¹H NMR (400 MHz, CHLOROFORM-d) δ 7.70-7.63 (m, 1H), 7.60 (d, J=0.8 Hz, 1H), 7.47-7.37 (m, 2H), 7.10-6.96 (m, 3H).

Step 2, 3-(4-chlorophenoxy)-5-fluoroaniline: To a solution of 1-(4-chlorophenoxy)-3-fluoro-5-nitrobenzene (500 mg, 1.9 μmol, 1.0 equiv.) in ethanol (5 mL) was added NH₄Cl (500 mg, 9.3 μmol, 5.0 equiv.) in water (2.5 mL) and iron(0) (522 mg, 9.3 μmol, 5.0 equiv.). The mixture was stirred at 80° C. for 2 hours. The reaction mixture was concentrated under reduced pressure. The residue was diluted with water (20 mL) and extracted with ethyl acetate (3×15 mL). The combined organic layers were washed with brine (2×5 mL), dried over Na₂SO₄, filtered and concentrated under reduced pressure to afford the title compound (420 mg, 94% yield) as a white solid. ¹H NMR (400 MHz, CHLOROFORM-d) δ 7.31 (d, J=8.8 Hz, 2H), 6.98 (d, J=8.8 Hz, 2H), 6.07 (d, J=1.5 Hz, 3H), 4.59-2.48 (m, 2H).

### Example 1

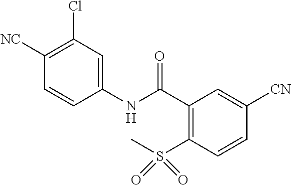 [SMILES: CS(=O)(=O)c1ccc(C#N)cc1C(=O)Nc1ccc(C#N)c(Cl)c1]

### N-(3-chloro-4-cyanophenyl)-5-cyano-2-(methylsulfonyl)benzamide

N-(3-chloro-4-cyanophenyl)-5-cyano-2-(methylsulfonyl)benzamide: To a mixture of 4-amino-2-chlorobenzonitrile (80 mg, 524 μmol, 1.0 equiv.) in isopropyl acetate (3.2 mL) was added 5-cyano-2-methylsulfonyl-benzoyl chloride (153 mg, 629 μmol, 1.2 equiv.) in isopropyl acetate (3.2 mL). The mixture was degassed and purged with N₂ and then stirred at 80° C. for 16 hours under N₂. The reaction mixture was diluted with water (20 mL) and extracted with ethyl acetate (3×10 mL). The combined organic layers were washed with brine (3×10 mL), dried over Na₂SO₄, filtered, and concentrated under reduced pressure. The reaction mixture was purified by preparative HPLC column (column: Phenomenex Luna C_{18 75×30} mm×3 um; mobile phase: 20%-60% acetonitrile in water (+formic acid)) to afford the title compound (96.6 mg, 50.4% yield) as a white solid. LCMS [M−1]=358.0. ¹H NMR (400 MHz, DMSO-d₆) δ 11.36 (br s, 1H), 8.43 (d, J=1.3 Hz, 1H), 8.31 (dd, J=1.5, 8.1 Hz, 1H), 8.21 (d, J=8.3 Hz, 1H), 8.09 (d, J=1.8 Hz, 1H), 7.99 (d, J=8.5 Hz, 1H), 7.68 (dd, J=1.9, 8.6 Hz, 1H), 3.44 (s, 3H).

The compounds in Table 1 were prepared following Scheme 1 using similar procedures to those described for Example 1.

TABLE 1

| | | | | LCMS |
|---|---|---|---|---|
| Example | | IUPAC | | Exact |
| number | Structure | Name | ¹H NMR | Mass |
| 2 | 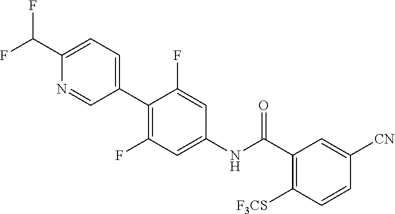 | 5-cyano-N-(4- (6- (difluoromethyl) pyridin-3-yl)- 3,5- difluorophenyl)- 2- ((trifluorometh- yl)thio)benzamide | (400 MHz, METHANOL- d₄) δ 8.76 (br s, 1H), 8.21 (s, 1H), 8.13 (br d, J = 8.0 Hz, 1H), 8.07- 7.97 (m, 2H), 7.83 (br d, J = 8.8 Hz, 1H), 7.58 (br d, J = 9.4 Hz, 2H), 6.80 (t, J = 55.2 Hz, 1H) | Calculated [M − 1] 484.0, found [M − 1] 484.1 |
| 3 | 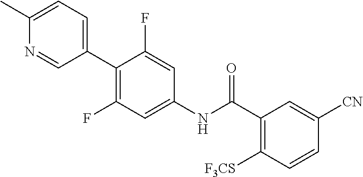 | 5-cyano-N- (3,5-difluoro- 4-(6- methylpyridin- 3-yl)phenyl)- 2- ((trifluorometh- yl)thio)benzamide | (400 MHz, METHANOL- d₄) 8.94 (s, 1H), 8.66 (d, J = 8.6 Hz, 1H), 8.20 (d, J = 1.2 Hz, 1H), 8.09- 7.98 (m, 3H), 7.64 (d, J = 10.2 Hz, 2H), 2.87 (s, 3H) | Calculated [M + 1] 450.1, found [M − 1] 450.1 |
| 4 | 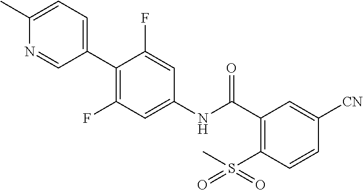 [SMILES: Cc1ccc(-c2c(F)cc(NC(=O)c3cc(C#N)ccc3S(C)(=O)=O)cc2F)cn1] | 5-cyano-N- (3,5-difluoro- 4-(6- methylpyridin- 3-yl)phenyl)- 2- (methylsulfonyl) benzamide | (400 MHz, METHANOL- d₄) δ 8.50 (s, 1H), 8.27 (d, J = 8.0 Hz, 1H), 8.17 (d, J = 1.4 Hz, 1H), 8.13 (dd, J = 1.6, 8.2 Hz, 1H), 7.85 (br d, J = 8.0 Hz, 1H), 7.50 (d, J = 9.8 | Calculated [M + 1] 428.1, found [M − 1] 428.1 |
| | | | Hz, 2H), 7.44 | |
| | | | (d, J = 8.0 Hz, | |
| | | | 1H), 3.41 (s, | |
| | | | 3H), 2.60 (s, | |
| | | | 3H). | |
| 5 | 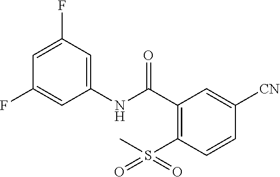 [SMILES: CS(=O)(=O)c1ccc(C#N)cc1C(=O)Nc1cc(F)cc(F)c1] | 5-cyano-N- (3,5- difluorophenyl)- 2- (methylsulfonyl) benzamide | (400 MHz, DMSO-d₆) δ 11.13-11.11 (m, 1H), 8.40 (d, J = 1.4 Hz, 1H), 8.32- 8.28 (m, 1H), 8.20 (d, J = 8.1 Hz, 1H), 7.37 (dd, J = 2.0, 9.2 Hz, | Calculated [M − 1] 335.0, found [M − 1] 335.1 |
| | | | 2H), 7.07- | |
| | | | 7.00 (m, 1H), | |
| | | | 3.44 (s, 3H) | |
| 6 | 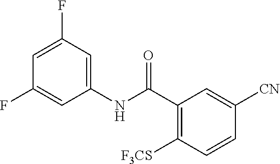 | 5-cyano-N- (3,5- difluorophenyl)- 2- (trifluorometh- ylsulfanyl)ben- zamide | (400 MHz, METHANOL- d₄) δ 8.17 (d, J = 1.4 Hz, 1H), 8.08-7.94 (m, 2H), 7.37 (dd, J = 2.2, 9.2 Hz, 2H), 6.77 (tt, J = 2.2, 9.2 Hz, 1H) | Calculated [M + 1] 359.0, found [M + 1] 359.0 |
| 7 | 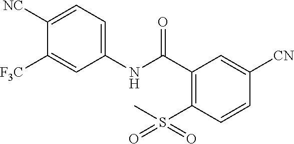 [SMILES: CS(=O)(=O)c1ccc(C#N)cc1C(=O)Nc1ccc(C#N)c(C(F)(F)F)c1] | 5-cyano-N-(4- cyano-3- (trifluorometh- yl)phenyl)-2- (methylsulfonyl) benzamide | (400 MHz, METHANOL- d₄ ) δ 8.35- 8.24 (m, 2H), 8.18 (dd, J = 1.6, 18.0 Hz, 2H), 8.00 (s, 2H), 3.39 (s, 3H) | Calculated [M − 1] 392.0, found [M −1] 392.1 |
| 8 | 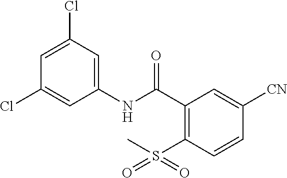 [SMILES: CS(=O)(=O)c1ccc(C#N)cc1C(=O)Nc1cc(Cl)cc(Cl)c1] | 5-cyano-N- (3,5- dichlorophenyl)- 2- (methylsulfonyl) benzamide | (400 MHz, METHANOL- d₄) δ 8.26 (d, J = 8.2 Hz, 1H), 8.17-8.09 (m, 2H), 7.69 (d, J = 1.8 Hz, 2H), 7.23 (t, J = 1.8 Hz, 1H), 3.40 (s, 3H) | Calculated [M + 1] 369.0, found [M + 1] 369.0 |
| 9 | 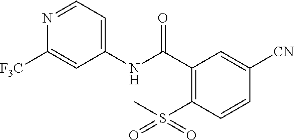 [SMILES: CS(=O)(=O)c1ccc(C#N)cc1C(=O)Nc1ccnc(C(F)(F)F)c1] | 5-cyano-2- (methylsulfonyl)- N-(2- (trifluorometh- yl)pyridin-4- yl)benzamide | (400 MHz, METHANOL- d₄) δ 8.62 (d, J = 5.6 Hz, 1H), 8.28 (d, J = 8.2 Hz, 1H), 8.24- 8.12 (m, 3H), 7.85 (dd, J = 1.8, 5.6 Hz, | Calculated [M + 1] 370.0, found [M + 1] 370.3 |
| | | | 1H), 3.40 (s, | |
| | | | 3H) | |
| 10 | 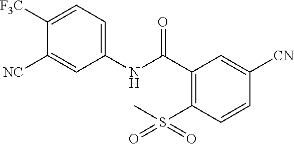 [SMILES: CS(=O)(=O)c1ccc(C#N)cc1C(=O)Nc1ccc(C(F)(F)F)c(C#N)c1] | 5-cyano-N-(3- cyano-4- (trifluorometh- yl)phenyl)-2- (methylsulfonyl) benzamide | (400 MHz, DMSO-d₆) δ 11.47 (s, 1H), 8.45 (d, J = 1.6 Hz, 1H), 8.37-8.29 (m, 2H), 8.22 (d, J = 8.2 Hz, 1H), 8.14-7.99 (m, | Calculated [M − 1] 392.0, found [M − 1] 392.1 |
| | | | 2H), 3.45 (s, | |
| | | | 3H) | |
| 11 | 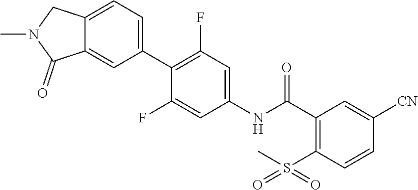 [SMILES: CN1Cc2ccc(-c3c(F)cc(NC(=O)c4cc(C#N)ccc4S(C)(=O)=O)cc3F)cc2C1=O] | 5-cyano-N- (3,5-difluoro- 4-(2-methyl-3- oxoisoindolin- 5-yl)phenyl)- 2- (methylsulfonyl) benzamide | (400 MHz, DMSO-d₆) δ 11.23 (s, 1H), 8.43 (d, J = 1.4 Hz, 1H), 8.31 (dd, J = 1.6, 8.2 Hz, 1H), 8.22 (d, J = 8.2 Hz, 1H), 7.73- 7.70 (m, 2H), 7.69-7.65 (m, 1H), 7.56- 7.48 (m, 2H), 4.53 (s, 2H), 3.47 (s, 3H), 3.11 (s, 3H) | Calculated [M + 1] 482.1, found [M + 1] 482.1 |
| 12 | 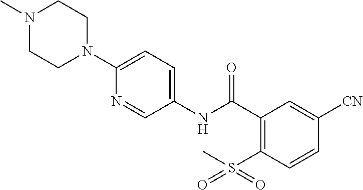 [SMILES: CN1CCN(c2ccc(NC(=O)c3cc(C#N)ccc3S(C)(=O)=O)cn2)CC1] | 5-cyano-N-(6- (4- methylpiperazin- 1-yl)pyridin- 3-yl)-2- (methylsulfonyl) benzamide | (400 MHz, DMSO-d₆) δ 10.61 (s, 1H), 8.35 (dd, J = 2.2, 3.8 Hz, 2H), 8.28- 8.24 (m, 1H), 8.19-8.15 (m, 2H), 7.85 (dd, J = 2.6, 9.2 Hz, 1H), 6.90 (d, J = 9.0 Hz, | Calculated [M + 1] 400.1, found [M + 1] 400.1 |
| | | | 1H), 3.44 (s, | |
| | | | 3H), 3.51 (m, | |
| | | | 4H), 2.60- | |
| | | | 2.54 (m, 4H), | |
| | | | 2.33 (s, 3H) | |
| 13 | 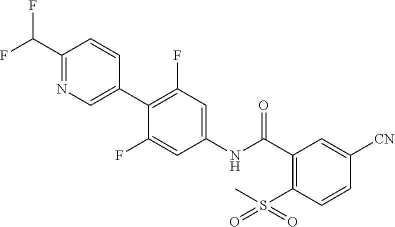 [SMILES: CS(=O)(=O)c1ccc(C#N)cc1C(=O)Nc1cc(F)c(-c2ccc(C(F)F)nc2)c(F)c1] | 5-cyano-N-(4- (6- (difluoromethyl) pyridin-3-yl)- 3,5- difluorophenyl)- 2- (methylsulfonyl) benzamide | (400 MHz, METHANOL- d₄) δ 8.76 (s, 1H), 8.27 (d, J = 8.0 Hz, 1H), 8.20-8.11 (m, 3H), 7.84 (d, J = 8.0 Hz, 1H), 7.54 (d, J = 10.0 Hz, 2H), 6.96-6.64 (m, 1H), 3.41 (s, 3H) | Calculated [M + 1] 464.1, found [M + 1] 464.0 |
| 14 | 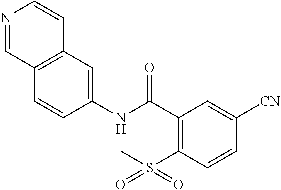 [SMILES: CS(=O)(=O)c1ccc(C#N)cc1C(=O)Nc1ccc2cnccc2c1] | 5-cyano-N- (isoquinolin-6- yl)-2- (methylsulfonyl) benzamide | (400 MHz, METHANOL- d₄) δ 9.17 (s, 1H), 8.48 (d, J = 1.4 Hz, 1H), 8.40 (d, J = 5.8 Hz, 1H), 8.29 (d, J = 8.0 Hz, 1H), 8.21 (d, J = 1.4 Hz, 1H), 8.18-8.08 (m, 2H), 7.86- | Calculated [M + 1] 352.1, found [M + 1] 351.9 |
| | | | 7.77 (m, 2H), | |
| | | | 3.43 (s, 3H) | |
| 15 | 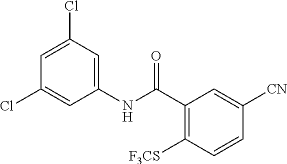 | 5-cyano-N- (3,5- dichlorophenyl)- 2- ((trifluorometh- yl)thio)benzamide | (400 MHz, METHANOL- d₄) δ 8.17 (d, J = 1.4 Hz, 1H), 8.06-7.90 (m, 2H), 7.72 (d, J = 1.8 Hz, 2H), 7.25 (t, J = 1.8 Hz, 1H) | Calculated [M + 1] 391.0, found [M + 1] 391.0 |
| 16 | 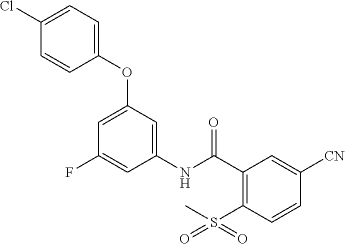 [SMILES: CS(=O)(=O)c1ccc(C#N)cc1C(=O)Nc1cc(F)cc(Oc2ccc(Cl)cc2)c1] | N-(3-(4- chlorophenoxy)- 5- fluorophenyl)- 5-cyano-2- (methylsulfonyl) benzamide | (400 MHz, CHLOROFORM- d) δ 8.37 (s, 1H), 8.00 (s, 1H), 7.92- 7.83 (m, 2H), 7.36 (d, J = 8.8 Hz, 2H), 7.24 (t, J = 2.0 Hz, 1H), 7.07- 6.98 (m, 2H), 6.82 (s, 1H), 6.45 (br d, J = 9.8 Hz, 1H), 3.34 (s, 3H) | Calculated [M − 1] 443.0, found [M − 1] 443.0 |
| 17 | 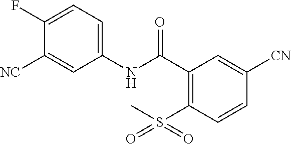 [SMILES: CS(=O)(=O)c1ccc(C#N)cc1C(=O)Nc1ccc(F)c(C#N)c1] | 5-cyano-N-(3- cyano-4- fluorophenyl)- 2- (methylsulfonyl) benzamide | (400 MHz, DMSO-d₆) δ 11.13 (s, 1H), 8.40 (d, J = 1.2 Hz, 1H), 8.30 (dd, J = 1.4, 8.2 Hz, 1H), 8.25-8.12 (m 2H), 7.92 | Calculated [M + 1] 344.0, found [M + 1] 344.0 |
| | | | (ddd, J = 2.6, | |
| | | | 4.8, 9.2 Hz, | |
| | | | 1H), 7.59 (t, J = | |
| | | | 9.0 Hz, 1H), | |
| | | | 3.45 (s, 3H) | |
| 18 | 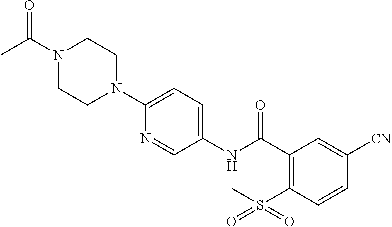 [SMILES: CC(=O)N1CCN(c2ccc(NC(=O)c3cc(C#N)ccc3S(C)(=O)=O)cn2)CC1] | N-(6-(4- acetylpiperazin- 1-yl)pyridin- 3-yl)-5-cyano- 2- (methylsulfonyl) benzamide | (400 MHz, METHANOL- d₄) δ 8.66 (d, J = 2.4 Hz, 1H), 8.28 (d, J = 8.0 Hz, 1H), 8.19- 8.12 (m, 2H), 8.09 (dd, J = 2.4, 9.8 Hz, 1H), 7.47 (d, J = 9.8 Hz, 1H), 3.93-3.80 (m, 6H), 3.79- 3.71 (m, 2H), | Calculated [M + 1] 428.1, found [M + 1] 428.1 |
| | | | 3.39 (s, 3H), | |
| | | | 2.18 (s, 3H) | |
| 19 | 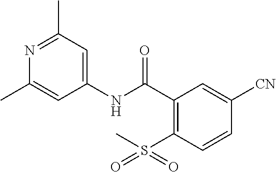 [SMILES: Cc1cc(NC(=O)c2cc(C#N)ccc2S(C)(=O)=O)cc(C)n1] | 5-cyano-N- (2,6- dimethylpyridin- 4-yl)-2- (methylsulfonyl) benzamide | (400 MHz, METHANOL- d₄) δ 8.29 (d, J = 8.2 Hz, 1H), 8.23 (d, J = 1.4 Hz, 1H), 8.21- 8.17 (m, 1H), 7.88 (s, 2H), 3.38 (s, 3H), 2.71 (s, 6H) | Calculated [M + 1] 330.1, found [M + 1] 330.0 |
| 20 | 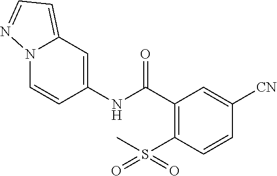 [SMILES: CS(=O)(=O)c1ccc(C#N)cc1C(=O)Nc1ccn2nccc2c1] | 5-cyano-2- (methylsulfonyl)- N- (pyrazolo[1,5- a]pyridin-5- yl)benzamide | (400 MHz, METHANOL- d₄) δ 8.49 (d, J = 7.4 Hz, 1H), 8.27 (d, J = 8.2 Hz, 1H), 8.19 (dd, J = 1.8, 9.2 Hz, 2H), 8.13 (dd, J = 1.8, 8.1 Hz, 1H), 7.93 (d, J = | Calculated [M + 1] 341.1, found [M + 1] 341.0 |
| | | | 2.4 Hz, 1H), | |
| | | | 7.03 (dd, J = | |
| | | | 2.4, 7.5 Hz, | |
| | | | 1H), 6.55 (d, J = | |
| | | | 2.0 Hz, 1H), | |
| | | | 3.42 (s, 3H) | |
| 21 | 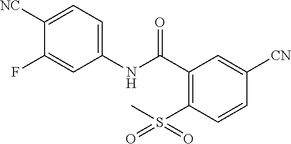 [SMILES: CS(=O)(=O)c1ccc(C#N)cc1C(=O)Nc1ccc(C#N)c(F)c1] | 5-cyano-N-(4- cyano-3- fluorophenyl)- 2- (methylsulfonyl) benzamide | (400 MHz, DMSO-d₆) δ 11.49-11.34 (br s, 1H), 8.52- 8.39 (m, 1H), 8.32 (dd, J = 1.6, 8.0 Hz, 1H), 8.22 (d, J = 8.4 Hz, 1H), | Calculated [M + 1] 344.1, found [M + 1] 344.1 |
| | | | 7.99-7.81 (m, | |
| | | | 2H), 7.56 (dd, | |
| | | | J = 1.8, 8.6 | |
| | | | Hz, 1H), 3.44 | |
| | | | (s, 3H) | |
| 22 | 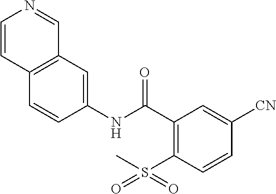 [SMILES: CS(=O)(=O)c1ccc(C#N)cc1C(=O)Nc1ccc2ccncc2c1] | 5-cyano-N- (isoquinolin-7- yl)-2- (methylsulfonyl) benzamide | (400 MHz, METHANOL- d₄) δ 9.20 (s, 1H), 8.60- 8.57 (m, 1H), 8.39 (d, J = 5.6 Hz, 1H), 8.29 (d, J = 8.2 Hz, 1H), 8.21 (d, J = 1.6 Hz, 1H), 8.14 (dd, J = 1.6, 8.2 Hz, | Calculated [M + 1] 352.1, found [M + 1] 352.1 |
| | | | 1H), 8.00- | |
| | | | 7.91 (m, 2H), | |
| | | | 7.81 (d, J = 5.8 | |
| | | | Hz, 1H), 3.44 | |
| | | | (s, 3H) | |
| 23 | 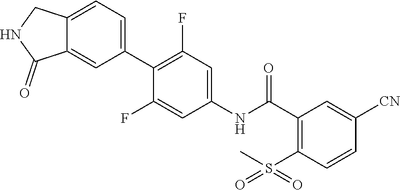 [SMILES: CS(=O)(=O)c1ccc(C#N)cc1C(=O)Nc1cc(F)c(-c2ccc3c(c2)C(=O)NC3)c(F)c1] | 5-cyano-N- (3,5-difluoro- 4-(3- oxoisoindolin- 5-yl)phenyl)- 2- (methylsulfonyl) benzamide | (400 MHz, DMSO-d₆) δ 11.23 (s, 1H), 8.68 (s, 1H), 8.43 (d, J = 1.4 Hz, 1H), 8.31 (dd, J = 1.6, 8.2 Hz, 1H), 8.22 (d, J = 8.2 Hz, 1H), 7.78- 7.65 (m, 3H), 7.52 (br d, J = | Calculated [M + 1] 468.1, found [M + 1] 468.0 |
| | | | 9.6 Hz, 2H), | |
| | | | 4.45 (s, 2H), | |
| | | | 3.46 (s, 3H) | |
| 24 | 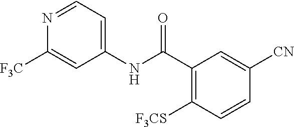 | 5-cyano-N-(2- (trifluorometh- yl)pyridin-4- yl)-2- ((trifluorometh- yl)thio)benzamide | (400 MHz, DMSO-d₆) δ 11.43 (br s, 1H), 8.73 (d, J = 5.6 Hz, 1H), 8.47 (d, J = 1.8 Hz, 1H), 8.16- 8.23 (m, 2H), | Calculated [M + 1] 392.0, found [M + 1] 392.0 |
| | | | 8.05 (d, J = 8.4 | |
| | | | Hz, 1H), 7.91 | |
| | | | (dd, J = 5.6, | |
| | | | 1.88 Hz, 1 H) | |
| 25 | 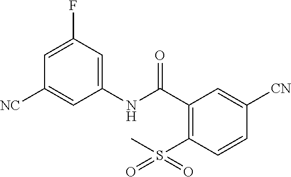 [SMILES: CS(=O)(=O)c1ccc(C#N)cc1C(=O)Nc1cc(F)cc(C#N)c1] | 5-cyano-N-(3- cyano-5- fluorophenyl)- 2- (methylsulfonyl) benzamide | (400 MHz, DMSO-d₆) δ 11.30 (s, 1H), 8.42 (d, J = 1.4 Hz, 1H), 8.30 (d, J = 1.6 Hz, 1H), 8.22 (s, 1H), 7.87 (s, 1H), 7.84 (br d, J = 10.6 Hz, 1H), 7.71- | Calculated [M − 1] 342.0, found [M − 1] 342.1 |
| | | | 7.63 (m, 1H), | |
| | | | 3.45 (s, 3H) | |
| 26 | 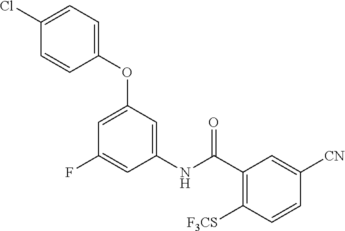 | N-(3-(4- chlorophenoxy)- 5- fluorophenyl)- 5-cyano-2- ((trifluorometh- yl)thio)benzamide | (400 MHz, DMSO-d₆) δ 10.91 (s, 1H), 8.36 (d, J = 1.4 Hz, 1H), 8.14 (dd, J = 1.8, 8.4 Hz, 1H), 7.99 (d, J = 8.2 Hz, 1H), 7.50 (d, J = 8.6 Hz, 2H), 7.45 (br d, J = 10.8 Hz, 1H), 7.17 (d, J = 8.8 Hz, 2H), | Calculated [M − 1] 465.0, found [M − 1] 465.1 |
| | | | 7.05 (s, 1H), | |
| | | | 6.76 (br d, J = | |
| | | | 10.0 Hz, 1H) | |
| 27 | 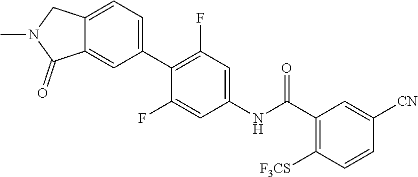 | 5-cyano-N- (3,5-difluoro- 4-(2-methyl-3- oxoisoindolin- 5-yl)phenyl)- 2- ((trifluorometh- yl)thio)benzamide | (400 MHz, DMSO-d₆) δ 11.22-11.15 (m, 1H), 8.43 (d, J = 1.2 Hz, 1H), 8.18 (dd, J = 1.8, 8.2 Hz, 1H), 8.04 (d, J = 8.2 Hz, 1H), 7.74- 7.69 (m, 2H), | calculated [M + 1] 504.1, found [M + 1] 504.1 |
| | | | 7.69-7.64 (m, | |
| | | | 1H), 7.60- | |
| | | | 7.51 (m, 2H), | |
| | | | 4.53 (s, 2H), | |
| | | | 3.08 (br s, 3H) | |
| 28 | 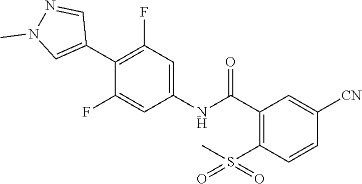 [SMILES: Cn1cc(-c2c(F)cc(NC(=O)c3cc(C#N)ccc3S(C)(=O)=O)cc2F)cn1] | 5-cyano-N- (3,5-difluoro- 4-(1-methyl- 1H-pyrazol-4- yl)phenyl)-2- (methylsulfonyl) benzamide | (400 MHz, DMSO-d₆) δ 11.14 (s, 1H), 8.40 (d, J = 1.4 Hz, 1H), 8.30 (dd, J = 8.2, 1.6 Hz, 1H), 8.20 (d, J = 8.2 Hz, 1H), 7.80 (s, 1H), 8.13 (s, 1H), 7.47 (d, J = 10.8 | Calculated [M + 1] 417.1, found [M + 1] 417.1 |
| | | | Hz, 2H), 3.92 | |
| | | | (s, 3H), 3.46 | |
| | | | (s, 3H) | |
| 29 | 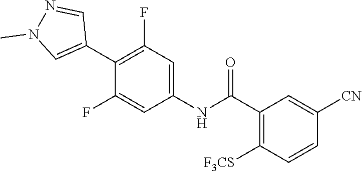 | 5-cyano-N- (3,5-difluoro- 4-(1-methyl- 1H-pyrazol-4- yl)phenyl)-2- ((trifluorometh- yl)thio)benzamide | (400 MHz, DMSO-d₆) δ 11.15 (s, 1H), 8.41 (d, J = 1.8 Hz, 1H), 8.08- 8.22 (m, 2H), 8.01 (d, J = 8.0 Hz, 1H), 7.80 (s, 1H), 7.53 (d, J = 10.8 Hz, 2H), | Calculated [M + 1] 439.1, found [M + 1] 439.1 |
| | | | 3.92 (s, 3H) | |
| 30 | 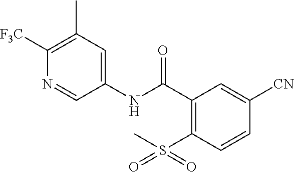 [SMILES: Cc1cc(NC(=O)c2cc(C#N)ccc2S(C)(=O)=O)cnc1C(F)(F)F] | 5-cyano-N-(5- methyl-6- (trifluorometh- yl)pyridin-3- yl)-2- (methylsulfonyl) benzamide | (400 MHz, METHANOL- d₄) δ 8.76- 8.70 (m, 1H), 8.28 (d, J = 8.2 Hz, 1H), 8.22 (s, 1H), 8.20 (d, J = 1.4 Hz, 1H), 8.14 (dd, J = 1.6, 8.2 Hz, 1H), 3.40 | Calculated [M + 1] 384.1, found [M + 1] 384.1 |
| | | | (s, 3H), 2.58- | |
| | | | 2.49 (m, 3H) | |

### Example 31

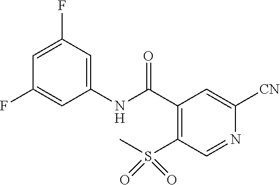 [SMILES: CS(=O)(=O)c1cnc(C#N)cc1C(=O)Nc1cc(F)cc(F)c1]

### 2-cyano-N-(3,5-difluorophenyl)-5-(methylsulfonyl)isonicotinamide

Sodium methanesulfinate (10.4 mg, 102 μmol, 1.5 equiv.) was added to a solution of 5-chloro-2-cyano-N-(3,5-difluorophenyl)isonicotinamide (20 mg, 68.1 μmol, 1.0 equiv.) in NMP (0.5 mL) at 0° C. Then the solution was stirred at 20° C. for 36 hours. The mixture was diluted with water (15 mL) and extracted with ethyl acetate (2×20 mL). The combined organic layers were washed with brine (2×15 mL), dried with anhydrous Na₂SO₄, filtered and concentrated under reduced pressure. The residue was purified by preparative TLC (3:1 petroleum ether:ethyl acetate) to afford the title compound (15.7 mg, 68% yield) as white solid. LCMS [M+1]=337.9. ¹H NMR (400 MHz, CHLOROFORM-d) δ 9.04-9.00 (m, 1H), 8.73 (s, 1H), 7.85 (s, 1H), 7.10 (br d, J=6.6 Hz, 2H), 6.57 (br t, J=8.8 Hz, 1H), 3.41 (s, 3H).

| Example | | | | LCMS |
|---|---|---|---|---|
| number | Structure | IUPAC Name | ¹H NMR | Exact Mass |
| 32 | 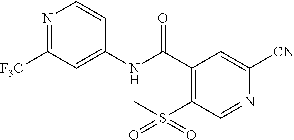 [SMILES: CS(=O)(=O)c1cnc(C#N)cc1C(=O)Nc1ccnc(C(F)(F)F)c1] | 2-cyano-5- (methylsulfonyl)- N-(2- (trifluoromethyl)py- ridin-4- yl)isonicotinamide | (400 MHz, DMSO- d₆) δ 11.67 (br s, 1H), 9.31 (s, 1H), 8.74 (d, J = 5.6 Hz, 1H), 8.70 (s, 1H), 8.13 (d, J = 1.8 Hz, 1H), 7.80 (dd, J = 1.8, 5.4 Hz, 1H), 3.51 (s, 3H) | Calculated [M + 1] 371.0, found [M − 1] 371.0 |

### Example 33

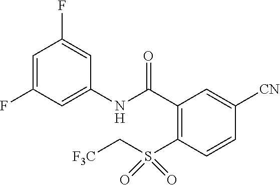 [SMILES: N#Cc1ccc(S(=O)(=O)CC(F)(F)F)c(C(=O)Nc2cc(F)cc(F)c2)c1]

### 5-cyano-N-(3,5-difluorophenyl)-2-((2,2,2-trifluoroethyl)sulfonyl)benzamide

Step 1, 5-cyano-N-(3,5-difluorophenyl)-2-iodo-benzamide: A mixture of 5-cyano-2-iodo-benzoic acid (1.0 g, 3.66 mmol, 1.0 equiv.) in SOCl₂ (10 mL) was degassed with N₂, and then the mixture was stirred at 100° C. for 2 hours under N₂. The mixture was concentrated under reduced pressure to afford 5-cyano-2-iodo-benzoyl chloride (1.0 g, crude) as light yellow solid. The crude product was used directly in next step. To a solution of 3,5-difluoroaniline (443 mg, 3.43 mmol, 1.0 equiv.) in isopropyl acetate (5 mL) was added a solution of 5-cyano-2-iodo-benzoyl chloride (1.0 g, 3.43 mmol, 1.0 equiv.) in isopropyl acetate (10 mL). The mixture was stirred at 80° C. for 16 hours. The reaction mixture was diluted with isopropyl acetate (10 mL), filtered and the filtered cake was washed by isopropyl acetate (3×10 mL). The solid was concentrated under reduced pressure. The residue was triturated with DCM at 25° C. for 30 minutes to afford the title compound (390 mg, 30% yield) as a white solid. LCMS [M+1]=385.1.

Step 2. 5-cyano-N-(3,5-difluorophenyl)-2-((2,2,2-trifluoroethyl)thio)benzamide: To a solution of 5-cyano-N-(3,5-difluorophenyl)-2-iodo-benzamide (200 mg, 520.7 μmol, 1.0 equiv.) in DMSO (2 mL) was added NaH (20.8 mg, 521 μmol, 1.0 equiv.; 60% dispersion in oil). The mixture was stirred at 20° C. for 0.5 hours and then CuI (9.9 mg, 52 μmol, 0.1 equiv.) and 2,2,2-trifluoroethanethiol (46.2 uL, 521 μmol, 1.0 equiv.) were added to the mixture. The reaction mixture was stirred at 100° C. for 4 hours. The reaction was quenched by the addition of saturated aqueous NH₄Cl (8 mL) and the aqueous layer was extracted with ethyl acetate (2×15 mL). The combined organic layers were dried over Na₂SO₄, filtered, and concentrated in vacuum. The residue was purified by silica gel column chromatography (6:1 to 5:1 petroleum ether:ethyl acetate) to afford the title compound (140 mg, 72% yield) as a white solid.

LCMS [M+1]=373.1. ¹H NMR (400 MHz, METHANOL-d₄) δ 7.96 (s, 1H), 7.84 (s, 2H), 7.41-7.32 (m, 2H), 6.74 (tt, J=2.4, 9.2 Hz, 1H), 3.93 (q, J=9.8 Hz, 2H).

Step 3,5-cyano-N-(3,5-difluorophenyl)-2-((2,2,2-trifluoroethyl)sulfonyl)benzamide: To a solution of 5-cyano-N-(3,5-difluorophenyl)-2-((2,2,2-trifluoroethyl)thio)benzamide (140 mg, 376.0 μmol, 1.0 equiv.) in DCM (6 mL) was added m-CPBA (458 mg, 2.26 mmol, 6.0 equiv.; 85% purity) at 0° C. The mixture was stirred at 40° C. for 16 hours. The reaction mixture was quenched by the addition of Na₂SO₃ (300 mg) in H₂O (10 mL) at 20° C. and stirred at 20° C. for 0.5 hours. Then the reaction mixture was extracted with DCM (2×10 mL). The combined organic layers were dried over Na₂SO₄, filtered, and concentrated under reduced pressure. The residue was purified by preparative HPLC (column: Phenomenex Luna C18 75×30 mm×3 um; mobile phase: 45-64% acetonitrile in water (+formic acid)) to afford the title compound (38.6 mg, 26% yield) as a white solid. LCMS [M+1]=404.9. ¹H NMR (400 MHz, DMSO-d₆) δ 11.27 (br s, 1H), 8.51 (br s, 1H), 8.40-8.20 (m, 2H), 7.38 (br d, J=4.6 Hz, 2H), 7.07 (br s, 1H), 5.33-4.91 (m, 2H).

| | | | | LCMS |
|---|---|---|---|---|
| Example | | | | Exact |
| number | Structure | IUPAC Name | ¹H NMR | Mass |
| 34 | 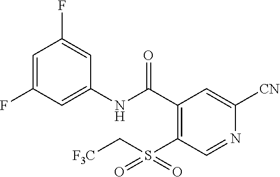 [SMILES: N#Cc1cc(C(=O)Nc2cc(F)cc(F)c2)c(S(=O)(=O)CC(F)(F)F)cn1] | 2-cyano-N-(3,5- difluorophenyl)-5- ((2,2,2- trifluoroethyl)sulfonyl) isonicotinamide | (400 MHz, DMSO- d₆) δ 11.41 (s, 1H), 9.33 (s, 1H), 8.76 (s, 1H), 7.35 (dd, J = 2.0, 9.0 Hz, 2H), 7.20- 7.06 (m, 1H), 5.18 (q, J = 9.8 Hz, 2H) | Calculated [M + 1] 406.0, found [M − 1] 406.0 |

### Biochemical and Cellular Assays

### PPARγ-NCOR1 Recruitment Assay:

Compound potency (EC₅₀) and maximal extent of NCOR1 recruitment to PPARG were assessed a TR-FRET binding assay measuring association of a biotinylated NCOR1 ID2 peptide (Biotin-GHSFADPASNLGLEDIIRKALMG-amide) to PPARG/RXRA LBD heterodimer. Specifically, a 20 microliters of TR-FRET master mix consisting of 2 nM WT PPARG LBD (e. coli expressed, His-TEV-Q203-Y477; Uniprot ID P37231-2), 2 nM WT RXRA LBD or mutant S427F RXRA LBD (e. coli expressed, Flag-TEV-E228-T462; P19793-1), 50 nM NCOR1, 80 nM Rosiglitazone, 25 nM streptavidin-d2 (Cisbio) and 0.3 nM Anti-His Tb (Cisbio) in 25 mM MOPS pH 7.4, 25 mM KCl, 1 mM EDTA, 0.01% BSA, 0.01% Tween-20 and 1 mM TCEP was added to 384-well plates containing duplicate 10-point dose response titrations of compounds in 60 nL DMSO (0.3% f.c. DMSO (v/v)). Mixtures were incubated for 3 hours and read in an EnVision plate reader (Perkin Elmer) with Ex/Em 615/665. To determine the potency (EC₅₀) and extent of NCOR1 recruitment, TR-FRET ratios were normalized to the average ratio of DMSO control wells (0%) and to the average maximum ratio for positive control compound (T0070907 (2-chloro-5-nitro-N-4-pyridinyl-benzamide); defined as 100%) in CDD Vault and analyzed using the Levenberg-Marquardt algorithm.

### PPARγ-MED1 Blockade Assay:

Compound potency (IC₅₀) and maximal extent of MED1 repulsion to PPARG were assessed a TR-FRET binding assay measuring association of a biotinylated MED1 LxxLL peptide (Biotin—VSSMAGNTKNHPMLMNLLKDNPAQ-amide) to PPARG/RXRA LBD heterodimer. Specifically, a 20 microliters of TR-FRET master mix consisting of 2 nM WT PPARG LBD (e. coli expressed, His-TEV-Q203-Y477; Uniprot ID P37231-2), 2 nM WT RXRA LBD (e. coli expressed, Flag-TEV-E228-T462; P19793-1), 350 nM NCOR1, 80 nM Rosiglitazone, 175 nM streptavidin-d2 (Cisbio) and 0.3 nM Anti-His Tb (Cisbio) in 25 mM MOPS pH 7.4, 25 mM KCl, 1 mM EDTA, 0.01% BSA, 0.01% Tween-20 and 1 mM TCEP was added to 384-well plates containing duplicate 10-point dose response titrations of compounds in 60 nL DMSO (0.3% DMSO f.c. (v/v)). Mixtures were incubated for 3 hours and read in an EnVision plate reader (Perkin Elmer) with Ex/Em 615/665. To determine the potency (IC₅₀) and extent of MED1 repulsion, TR-FRET ratios were normalized to the average ratio of DMSO control wells (0%) and to the average minimum ratio for positive control compound (GW9662 (2-chloro-5-nitrobenzanilide); defined as 100%) in CDD Vault and analyzed using the Levenberg-Marquardt algorithm.

### Bladder Cancer Pharmacodynamic Assay

5637 (PPARG amplified) and HT1197 (RXRA S427F mutation) cells were used for assessment of modulation of PPARG target genes using quantitative PCR. Cells were treated for 24 hours with PPARG inverse agonists prior to analysis of FABP4 (IDT, Cat: Hs.PT 58.20106818) and ANGPTL4 (IDT, Cat: Hs.PT 58.25480012) expression, with expression of the housekeeping gene TBP (IDT, Cat: Hs.PT 58v.39858774) used to normalize expression across samples. Quantitative PCR was performed using an ABI QuantStudio 7 Flex Reaction system. Data were analyzed and reported relative to DMSO control using the comparative Ct method (ΔΔCt).

### Table 1

For the PPARG-NCOR recruitment assay the EC₅₀ is expressed as follows, A: <10 nM, B: 10-100 nM, C: 100-1,000 nM, D: 1,000-10,000 nM, E: >10,000 nM. The % NCOR recruitment is expressed as follows, A: >100% (>the control compound, T907), B: <100% (<the control compound, T907).

For the PPARG-MED1 recruitment assay the EC₅₀ is expressed as follows, A: <10 nM, B: 10-100 nM, C: 100-1,000 nM, D: 1,000-10,000 nM, E: >10,000 nM. The % MED1 blockade is expressed as follows, A: >100% (>the control compound, GW9662), B: <100% (<the control compound, GW9662).

For the HT1197 cell assay the EC₅₀ is expressed as follows, A: <10 nM, B: 10-100 nM, C: 100-1,000 nM, D: 1,000-10,000 nM, E: >10,000 nM, ND: not determined. The % inhibition of ANGPTL4, a PPARG target gene, at 100 nM compound concentration is expressed as percentage of a DMSO control experiment.

| | PPARG - NCOR | PPARG - MED1 | HT1197 cell |
|---|---|---|---|
| | recruitment assay | blockade assay | assay EC₅₀ - |
| | EC₅₀ - % NCOR | EC₅₀ - % MED1 | % inhibition of |
| Example | recruitment | blockade | ANGPTL4 @ 100 |
| number | relative to T907 | relative to GW9662 | nM |
| 1 | C - A | C - A | 5.2 nM - 86% |
| 2 | C - A | C - A | NA - 83% |
| 3 | B - A | C - A | NA - 57% |
| 4 | C - A | C - A | NA - 74% |
| 5 | C - B | D - A | NA - 40% |
| 6 | C - B | C - A | NA - 54% |
| 7 | D - A | D - A | NA - 77% |
| 8 | C - B | C - A | NA - 41% |
| 9 | D - A | D - A | NA - 64% |
| 10 | C - B | C - A | NA - 48% |
| 11 | B - A | C - A | 1.3 nM - 79% |
| 12 | E - B | E - B | NA - 25% |
| 13 | C - A | C - A | 2.9 nM - 80% |
| 14 | D - B | D - B | NA - 35% |
| 15 | B - B | B - B | NA - 46% |
| 16 | B - B | B - B | NA - (−240%) |
| 17 | D - B | D - B | NA - 43% |
| 18 | E - B | E - B | NA - 13% |
| 19 | E - B | E - B | NA - 20% |
| 20 | E - B | E - B | NA - 0% |
| 21 | D - A | D - A | NA - 79% |
| 22 | E - B | E - B | NA - 0% |
| 23 | B - B | B - A | NA - 19% |
| 24 | D - A | D - A | NA - 49% |
| 25 | C - A | C - A | NA - 77% |
| 26 | C - B | C - B | NA (−160%) |
| 27 | C - A | C - A | NA - 79% |
| 28 | C - B | C - A | NA - 72% |
| 29 | C - B | C - A | NA - 65% |
| 30 | D - A | D - A | NA - 42% |
| 31 | B - A | B - A | NA - 72% |
| 32 | C - A | C - A | NA - 80% |
| 33 | D - B | D - B | NA - 19% |
| 34 | C - B | C - A | NA - 59% |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 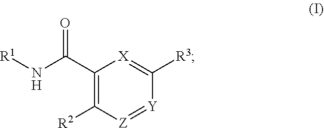
or a pharmaceutically acceptable salt thereof, wherein:
X, Y, and Z are each independently selected from N and —CR⁴;
R¹ is selected from phenyl, heterocyclyl, and heteroaryl, each of which are optionally substituted with 1 to 3 groups selected from R⁵;
R² is selected from halo, —SR^{a}, —SOR^{a}, —SO₂R, —OR, and —SO(═NR^{a})R^{b};
R³ is selected from cyano and nitro;
R⁴ is selected from hydrogen, halo, (C₁-C₄)alkyl, halo(C₁-C₄)alkyl, (C₁-C₄)alkoxy, and hydroxyl;
R⁵ is selected from halo, (C₁-C₄)alkyl, (C₁-C₄)alkoxy, halo(C₁-C₄)alkyl, halo(C₁-C₄)alkoxy, cyano, oxo, —(C₁-C₄)alkylOR^{a}, —(C₁-C₄)alkylC(O)R^{a}, —(C₁-C₄)alkylC(O)OR^{a}, —C(O)NR^{a}R^{b}, —(C₁-C₄)alkylC(O)NR^{a}R^{b}, —C(O)R^{a}, —C(O)OR^{a}, —NR^{a}R^{b}, —(C₁-C₄)alkylNR^{a}R^{b}, —C(O)NR^{a}SO₃H, —NR^{a}C(O)R^{b}, —NR^{a}C(O)OR^{b}, —NR^{a}C(S)OR^{b}, —NR^{c}C(O)N^{a}R^{b}, —NR^{c}C(S)NR^{a}R^{b}, —NRS(O)₂NR^{a}R^{b}, —C(S)R^{a}, —S(O)₂R^{a}, —S(O)R^{a}, —C(S)OR^{a}, —C(S)NR^{a}R^{b}, —NR^{a}C(S)R^{b}, —SR^{a}, —O(phenyl), phenyl, heterocyclyl, and heteroaryl, wherein each of said phenyl, heterocyclyl, heteroaryl, and the phenyl group on —O(phenyl) are optionally and independently substituted with 1 to 3 groups selected from R⁶;
R⁶ is selected from halo, (C₁-C₄)alkyl, halo(C₁-C₄)alkyl, (C₁-C₄)alkoxy, halo(C₁-C₄)alkoxy, nitro, oxo, cyano, —(C₁-C₄)alkylOR^{d}, —(C₁-C₄)alkylC(O)R^{d}, —(C₁-C₄)alkylC(O)OR^{d}, —C(O)NR^{d}R^{e}, —(C₁-C₄)alkylC(O)NR^{d}R^{e}, —C(O)R^{d}, —C(O)OR^{d}, —NR^{d}R^{e}, —(C₁-C₄)alkylNR^{d}R^{e}, —C(O)NR^{d}SO₃H, —NR^{d}C(O)R^{e}, —NR^{d}C(O)OR^{e}, —NR^{d}C(S)OR^{e}, —NR^{f}C(O)N^{d}R^{e}, —NR^{f}C(S)NR^{d}R^{e}, —NR^{f}S(O)₂NR^{d}R^{e}, —C(S)R^{d}, —S(O)₂R^{d}, —S(O)R^{d}, —C(S)OR^{d}, —C(S)NR^{d}R^{e}, —NR^{d}C(S)R^{e}, and —SR^{d}; and
R^{a}, R^{b}, R^{c}, R^{d}, and R^{e} are each independently selected from hydrogen, (C₁-C₄)alkyl, and halo(C₁-C₄)alkyl.

2. The compound of claim 1, or a pharmaceutically acceptable salt thereof, wherein X, Y, and Z are each —CR⁴; or X and Z are each —CR^{s} and Y is N.

3. The compound of claim 1, or a pharmaceutically acceptable salt thereof, wherein X, Y, and Z are each —CR⁴.

4. The compound of claim 1, or a pharmaceutically acceptable salt thereof, wherein R⁴ is selected from hydrogen, (C₁-C₄)alkyl, halo(C₁-C₄)alkyl, and (C₁-C₄)alkoxy.

5. The compound of claim 1, or a pharmaceutically acceptable salt thereof, wherein R⁴ is hydrogen.

6. The compound of claim 1, or a pharmaceutically acceptable salt thereof, wherein R³ is cyano.

7. The compound of claim 1, or a pharmaceutically acceptable salt thereof, wherein R² is selected from halo, —SR^{a}, —SOR^{a}, —SO₂R^{a}, and —SO(═NR^{a})R^{b}.

8. The compound of claim 1, or a pharmaceutically acceptable salt thereof, wherein R² is selected from —SO₂R^{a}, and —SR^{a}.

9. The compound of claim 1, or a pharmaceutically acceptable salt thereof, wherein R² is selected from —SO₂Me, SCF₃, —SO₂CH₂CF₃, and —SO₂CF₃.

10. The compound of claim 1, or a pharmaceutically acceptable salt thereof, wherein R¹ is selected from phenyl and heteroaryl, each of which are optionally substituted with 1 to 3 groups selected from R⁵.

11. The compound of claim 1, or a pharmaceutically acceptable salt thereof, wherein R¹ is selected from phenyl, pyridinyl, isoquinolinyl, pyrazolopyridinyl, each of which are optionally substituted with 1 to 3 groups selected from R⁵.

12. The compound of claim 1, or a pharmaceutically acceptable salt thereof, wherein R⁵ is selected from halo, oxo, cyano, (C₁-C₄)alkyl, (C₁-C₄)alkoxy, halo(C₁-C₄)alkyl, halo(C₁-C₄)alkoxy, —(C₁-C₄)alkylOR^{a}, —(C₁-C₄)alkylC(O)R^{a}, —(C₁-C₄)alkylC(O)OR^{a}, —C(O)NR^{a}R^{b}, —(C₁-C₄)alkylC(O)NR^{a}R^{b}, —C(O)R^{a}, —C(O)OR^{a}, —NR^{a}R^{b}, —(C₁-C₄)alkylNR^{a}R^{b}, NR^{a}C(O)R^{b}, —NR^{a}C(O)OR^{b}, —NR^{c}C(O)N^{a}R^{b}, —NR^{c}S(O)₂NR^{a}R^{b}, —S(O)₂R^{a}, —S(O)R^{a}, —SR^{a}, —O(phenyl), phenyl, heterocyclyl, and heteroaryl, wherein each of said phenyl, heterocyclyl, heteroaryl, and the phenyl group on —O(phenyl) are optionally and independently substituted with 1 to 3 groups selected from R.

13. The compound of claim 1, or a pharmaceutically acceptable salt thereof, wherein R⁵ is selected from halo, halo(C₁-C₄)alkyl, cyano, oxo, —O(phenyl), heterocyclyl, and heteroaryl, wherein each of said heterocyclyl, heteroaryl, and the phenyl group on —O(phenyl) are optionally and independently substituted with 1 to 3 groups selected from R⁶.

14. The compound of claim 1, or a pharmaceutically acceptable salt thereof, wherein R⁵ is selected from cyano, chloro, fluoro, CF₃, oxo, methyl, pyridinyl, piperazinyl, pyrazolyl, and —O(phenyl).

15. The compound of claim 1, or a pharmaceutically acceptable salt thereof, wherein R⁶ is selected from halo, (C₁-C₄)alkyl, halo(C₁-C₄)alkyl, (C₁-C₄)alkoxy, halo(C₁-C₄)alkoxy, oxo, cyano, —(C₁-C₄)alkylOR^{d}, —(C₁—C₄)alkylC(O)OR^{d}, —NR^{d}R^{e}, —(C₁-C₄)alkylNR^{d}R^{e}, —C(O)NR^{d}R^{e}, —(C₁-C₄)alkylC(O)NR^{d}R^{e}, —C(O)R^{d}, —C(O)OR^{d}, —S(O)₂R^{d}, —S(O)R^{d}, and —SR^{d}.

16. The compound of claim 1, or a pharmaceutically acceptable salt thereof, wherein R⁶ is selected from halo, (C₁-C₄)alkyl, halo(C₁-C₄)alkyl, oxo, —C(O)R^{d}.

17. The compound of claim 1, or a pharmaceutically acceptable salt thereof, wherein R⁶ is selected from methyl, CF₃, —CHCF₂, oxo, chloro, and C(O)CH₃.

18. The compound of claim 1, wherein the compound is selected from 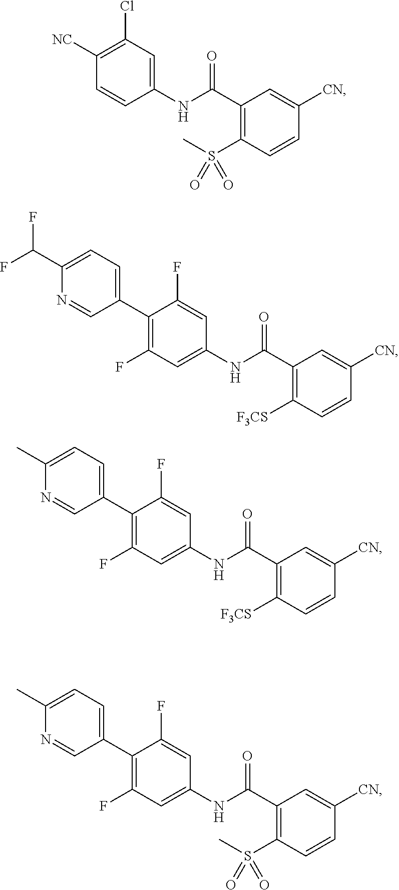 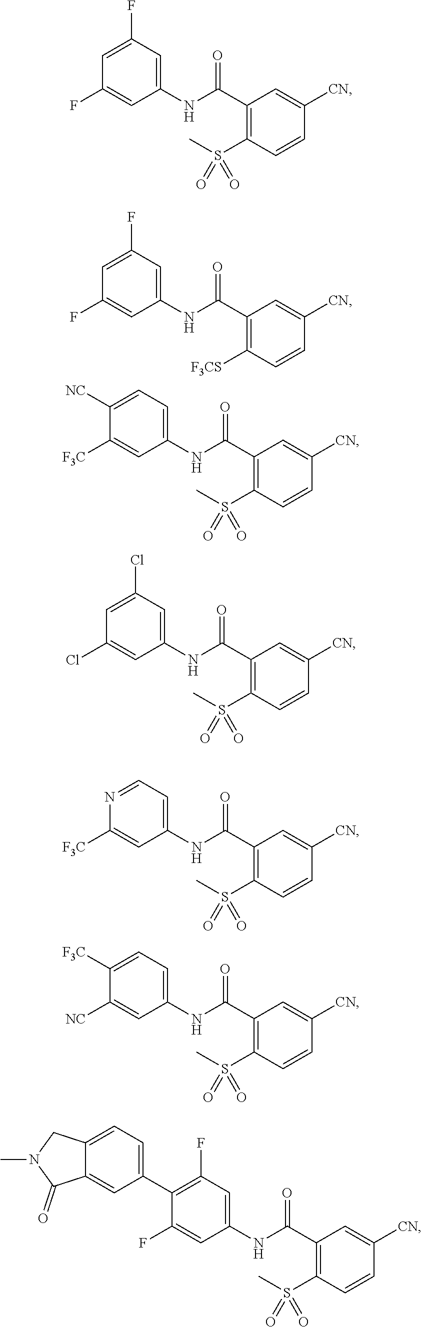 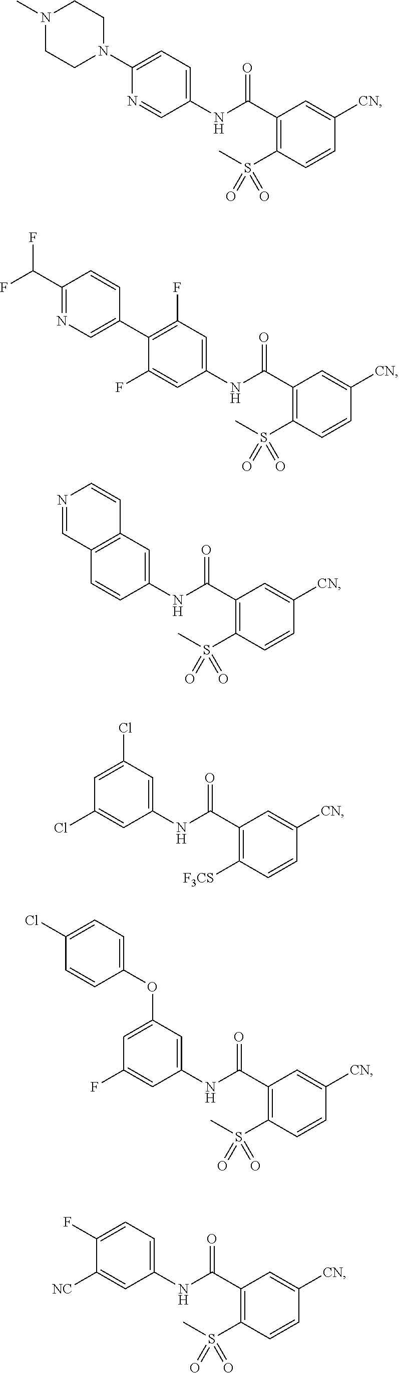 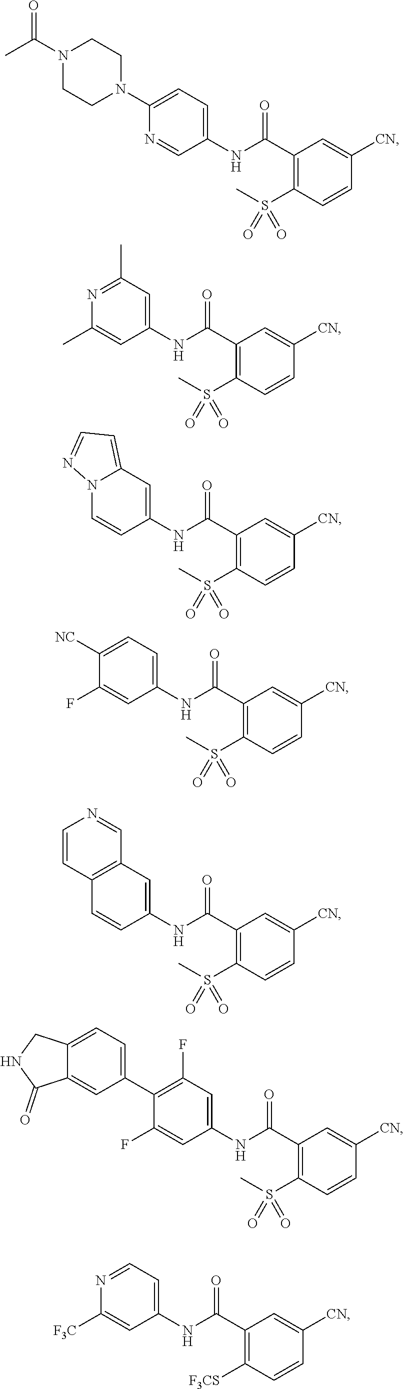 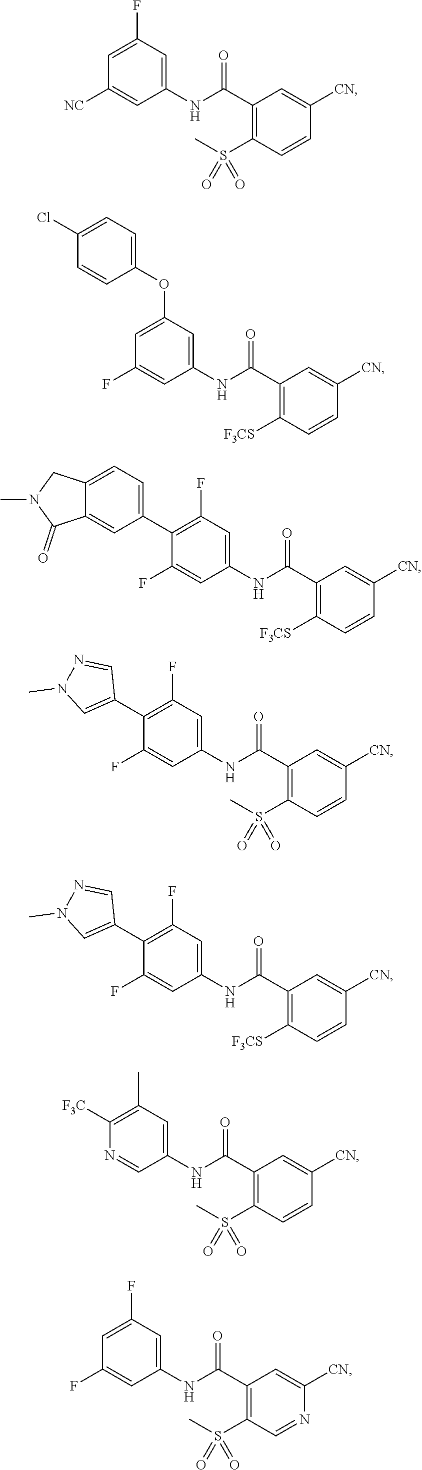 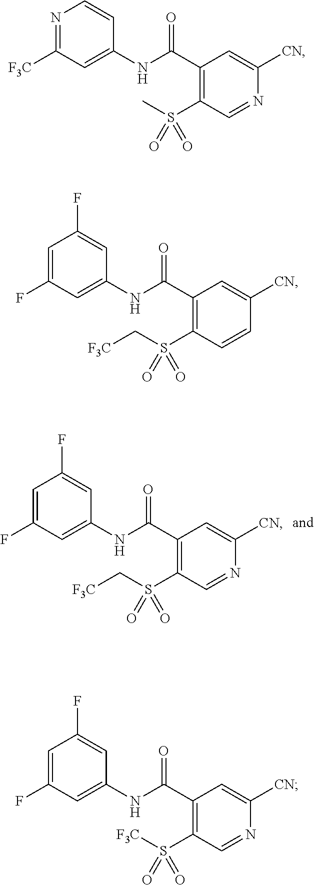 [SMILES: CS(=O)(=O)c1cnc(C#N)cc1C(=O)Nc1ccnc(C(F)(F)F)c1 ; N#Cc1ccc(S(=O)(=O)CC(F)(F)F)c(C(=O)Nc2cc(F)cc(F)c2)c1 ; N#Cc1cc(C(=O)Nc2cc(F)cc(F)c2)c(S(=O)(=O)CC(F)(F)F)cn1 ; N#Cc1cc(C(=O)Nc2cc(F)cc(F)c2)c(S(=O)(=O)C(F)(F)F)cn1]
or a pharmaceutically acceptable salt of any of the foregoing.

19. A pharmaceutical composition comprising a compound of claim 1, or a pharmaceutically acceptable salt thereof, and a pharmaceutically acceptable carrier.

20. A method of treating a cancer responsive to the suppression of PPARG in a subject, comprising administering to the subject a therapeutically effective amount of a compound of claim 1, or a pharmaceutically acceptable salt thereof.

21. The method of claim 20, wherein the cancer is selected from breast cancer, pancreatic cancer, ovarian cancer, prostate cancer, renal cancer, bladder cancer, testicular cancer, urothelial cancer, skin cancer, melanoma, colon cancer, kidney cancer, brain cancer and a hematopoietic cancer.

22. The method of claim 20, wherein the cancer is bladder cancer.
